(19) United States
(12) Patent Application Publication
(10) Pub. No.: US 2008/0066196 A1
(43) Pub. Date: Mar. 13, 2008

(54) SHORT INTERFERING RNA DUPLEXES TARGETING AN IRES SEQUENCE AND USES THEREFOR

(75) Inventor: Gene W. Lee, Chelmsford, MA (US)

Correspondence Address: FITZPATRICK CELLA (WYETH), 30 ROCKEFELLER PLAZA, NEW YORK, NY, 10112-3800, US

(73) Assignee: WYETH, Madison, NJ (US)

(21) Appl. No.: 11/737,488

(22) Filed: Apr. 19, 2007

Related U.S. Application Data
(60) Provisional application No. 60/792,968, filed on Apr. 19, 2006.

(51) Int. Cl. A01H 5/00; A01K 67/00; C12N 1/20; C12N 15/00; C12N 15/11; C12N 15/87; C12N 5/06

(52) U.S. Cl. 800/13; 435/243; 435/320.1; 435/325; 435/455; 536/24.5; 800/298

(57) ABSTRACT

The invention provides novel inhibitory polynucleotides directed against IRES sequences. The invention also provides genetically engineered expression vectors, host cells, transgenic animals, and transgenic plants comprising the novel inhibitory polynucleotides of the invention. The invention additionally provides methods of using the inhibitory polynucleotides of the invention.

### RELATED APPLICATIONS

This application claims the benefit of priority to U.S. Provisional Patent Application No. 60/792,968, filed Apr. 19, 2006, which is hereby incorporated by reference herein in its entirety.

### BACKGROUND OF THE INVENTION

1. Field of the Invention

The present invention relates to the use of inhibitory polynucleotides, particularly short interfering RNA (siRNA) duplexes, that target an internal ribosome entry site in methods of inhibiting gene expression, e.g., screening assays.

2. Related Background Art

It is well known in the art that the transcription of a gene usually requires a promoter that is upstream of the gene, that transcription usually results in a monocistronic mRNA (i.e., an mRNA transcript that comprises only one protein-coding region), and that translation of the resulting monocistronic mRNA is usually initiated by a translation initiation complex in a cap-dependent mechanism that involves recognition of a 5′ terminal cap-structure on the monocistronic mRNA (see, e.g., Merrick and Hershey (1996) “The pathway and mechanism of eukaryotic protein synthesis.” In Translational Control, J. W. B. Hershey, M. B. Mathews, and N. Sonenberg, Eds. (Cold Spring Harbor Laboratory Press, Cold Spring Harbor, N.Y.), pp. 31-69). The translation initiation complex usually moves along the monocistronic mRNA until it reaches a first initiation codon (AUG), usually within 50-100 nucleotides of the cap-structure, whereby translation of the mRNA into protein would usually commence. In other words, translation of the mRNA into protein generally commences at the first initiation AUG codon. This canonical model of monocistronic transcription and translation, found in most eukaryotic and some prokaryotic cells, poses a problem in the utilization of recombinant DNA technology, e.g., for gene therapy, because it is sometimes advantageous to transfer and express multiple transgenes within a single host cell.

Early in the development of recombinant DNA technology, when an investigator was interested in expressing more than one protein in a single host cell, the genes to be transferred (transgenes), e.g., those encoding each protein of interest, were placed on different expression vectors, necessitating that the host cell be successfully modified with each such expression vector. As expected, modification of a host cell with more than one expression vector often proved difficult and laborious. Alternatively, a single expression vector comprising each transgene that encoded a protein(s) of interest was created such that the transcription of each transgene was controlled by its own individual promoter, i.e., several monocistronic mRNAs were transcribed from the single expression vector. However, the presence of several promoters within one expression vector often resulted in reduction or loss of expression over time, likely due to interference between the promoter sequences. These problems were solved by the discovery and subsequent utilization of internal ribosome entry sites (IRESes). An IRES is generally placed downstream of a protein-coding region, the translation of which is initiated by a first initiation codon. The sequence of an IRES allows protein translation to commence from an internal (i.e., second) initiation codon (AUG), i.e., an AUG codon downstream of the IRES and the first initiation AUG codon, and thus allows an mRNA transcript to be polycistronic, i.e., capable of comprising more than one protein-coding region.

To date, IRESes have been identified in the 5′ region of noncapped viral mRNAs, such as members of the Picornaviridae family, e.g., poliomyelitis virus (Pelletier et al. (1988) Mol. Cell. Biol. 8(3):1103-12), poliovirus (PV), encephalomyocarditis virus (EMCV) (Jang et al. (1988) J. Virol. 62(8):2636-43), and foot-and-mouth disease virus (FMDV) (reviewed in Belsham and Sonenberg (1996) Microbiol. Rev. 60(3):499-511; Robertson et al. (1999) RNA 5(9):1167-79; Jackson and Kaminski (1995) RNA 1(10):985-1000; Herman (1989) Trends Biochem. Sci. 14(6):219-22). IRESes have also been detected in transcripts from other viruses, such as VL30-type murine retrotransposons (Berlioz et al. (1995) J. Virol. 69(10):6400-07), cardiovirus, rhinovirus, aphthovirus, hepatitis C virus (HCV), and more recently, in mRNAs encoding the gag precursor of the Friend (FMLV) and Moloney (MOMLV) murine leukemia viruses (Berlioz and Darlix (1995) J. Virol. 69(4):2214-22; Vagner et al. (1995) J. Biol. Chem. 270(35):20376-83). The presence of IRESes in cellular RNAs has also been described. Examples of cellular mRNAs that comprise IRESes include those encoding immunoglobulin heavy-chain binding protein (BiP) (Macejak and Samow (1991) Nature 353:90-94); certain growth factors such as vascular endothelial growth factor (VEGF), fibroblast growth factor 2 and insulin-like growth factor (Teerink et al. (1995) Biochim. Biophys. Acta 1264(3):403-08; Vagner et al. (1995) Mol. Cell. Biol. 15(1):35-44); translational initiation factor eIF4G (Gan and Rhoads (1996) J. Biol. Chem. 271(2):623-26), and the yeast transcription factors TFIID and HAP4 (lizuka et al. (1994) Mol. Cell. Biol. 14(11):7322-30) (see also, Oh et al. (1992) Genes Dev. 6(9):1643-53; He et al. (1996) Proc. Natl. Acad. Sci. USA 93(14):7274-78; He et al. (1996) Gene 175(1-2):121-25; Tomanin et al. (1997) Gene 193(2):129-40; Gambotto et al. (1999) Cancer Gene Ther. 6(1):45-53; Qiao et el. (1999) Cancer Gene Ther. 6(4):373-79)).

In the context of recombinant DNA technology, expression vectors comprising IRESes have been described (see, e.g., International Published Patent Application Nos. WO 98/37189; WO 99/25860; and WO 93/03143). Generally, these expression vectors would allow the placement of an IRES between at least two transgenes, and subsequently would allow the expression of at least two transgenes from a single promoter. In particular, transcription from the single promoter would result in an mRNA that could be polycistronic, e.g., wherein the at least two protein-coding regions were separated by at least one IRES, and translation would begin at both the first initiation AUG codon, and an internal AUG codon(s) downstream of the IRES(es).

IRESes are powerful tools in the field of recombinant DNA technology because they allow the translation of several genes from a single mRNA transcript. In other words, use of an IRES for the expression of multiple different transgenes by a single host cell obviates the need to modify a host cell with either multiple expression vectors, or with an expression vector comprising several promoters that may interfere with one another. Additionally, several groups have reported the stability and functionality of the EMCV-IRES in chicken and mouse embryos, and in many organs of adult mice (Ghattas et al. (1991) Mol. Cell. Biol. 11(12):5848-59; Kim et al. (1992) Mol. Cell. Biol. 12(8):3636-43; Creancier et al. (2000) J. Cell. Biol. 150(1):275-81).

Although IRESes have been incorporated in recombinant DNA technology, the utility of this technology, e.g., in gene therapy, may be advanced upon investigation into 1) the effects of expression of such transgenes on the modified host cell or organism, e.g., the effect of the transgene on the metabolism of the modified host, and 2) the functions of the proteins encoded by transgenes. A popular method of investigating the effect(s) and function(s) of transgene expression on a host cell or organism is to inhibit (e.g., reduce, interfere, downregulate, knock down, etc.) expression of the transgene after it has been successfully introduced into and expressed by the host cell or organism.

Several approaches have been developed to inhibit the expression of a gene of interest (e.g., a transgene, endogenous gene, etc.), including antisense, triple-helix, cosuppression, and RNAi methods. These methods have involved the utilization of targeting nucleic acid molecules that are the reverse complement of the targeted gene mRNA transcript (or portions thereof), form triple-helical structures with the targeted gene, are exact duplicates of the targeted gene, or are duplex molecules of short interfering RNA (siRNA) comprising a nucleotide sequence of the targeted gene (or portions thereof), respectively. To date, these approaches have been used to specifically target a single gene of interest, and as such, require that the sequence of the targeting molecule (e.g., the antisense molecule, triple-helix forming molecule, the cosuppression transgene molecule, and the siRNA molecule) correspond to (i.e., specifically hybridize to at least a portion of one, the other, or both strands of) at least a portion of the targeted gene of interest. As such, the application of these approaches has heretofore required the investigator to know the sequence of the targeted gene of interest, and/or the portion of the targeted gene sequence that has the greatest susceptibility to being targeted, and to create a unique targeting molecule for each targeted gene. To date, there is neither a mechanism by which to inhibit expression of a targeted gene without first knowing the sequence of the gene of interest, nor, if the sequence of the gene of interest is known, is there an efficient assay to determine which portion of the gene sequence is more susceptible to inhibition.

The present invention solves these problems by providing inhibitory polynucleotides and methods of using these inhibitory polynucleotides in, methods of e.g., 1) inhibiting (e.g., reducing, interfering with, downregulating, knocking down, etc.) the expression of at least one transgene of interest that does not require the investigator to know or determine the sequence of the transgene and/or 2) screening libraries of targeting polynucleotides to inhibit expression of a gene of interest, regardless of whether the gene of interest is a transgene or an endogenous gene.

### SUMMARY OF THE INVENTION

The present invention is related to the discovery that inhibitory polynucleotides that target an IRES may be used to downregulate (e.g., inhibit) the expression of at least one gene of interest that is transcribed with its protein-coding region as part of an mRNA transcript comprising a nucleotide sequence corresponding to the targeted IRES. Accordingly, the present invention provides an inhibitory polynucleotide directed against an IRES, e.g., an IRES that has the nucleotide sequence of SEQ ID NO:1.

An inhibitory polynucleotide of the invention may be an siRNA molecule, e.g., in one embodiment of the invention, an inhibitory polynucleotide of the invention comprises a first strand of an siRNA. In another embodiment of the invention, the first strand of the siRNA has and/or consists essentially of the RNA equivalent of a nucleotide sequence selected from the group consisting of the nucleotide sequence of SEQ ID NO:1, a portion of the nucleotide sequence of SEQ ID NO:1, the complement of the nucleotide sequence of SEQ ID NO:1, and a portion of the complement of the nucleotide sequence of SEQ ID NO:1. In another embodiment of the invention, the first strand of the siRNA is between 5 and 548 nucleotides in length. In another embodiment of the invention, the first strand of the siRNA has and/or consists essentially of the RNA equivalent of a nucleotide sequence selected from the group consisting of the nucleotide sequence of SEQ ID NO:2, the nucleotide sequence of SEQ ID NO:3, the nucleotide sequence of SEQ ID NO:4, and the nucleotide sequence of subsequences thereof. In another embodiment of the invention, the first strand of the siRNA is self-complementary and further comprises a hairpin loop, e.g., an siRNA of the invention may comprise the RNA equivalent of a nucleotide sequence selected from the group consisting of the nucleotide sequence complementary to the nucleotide sequence of SEQ ID NO:2, the nucleotide sequence complementary to the nucleotide sequence of SEQ ID NO:3, and the nucleotide sequence complementary to the nucleotide sequence of SEQ ID NO:4. In yet another embodiment of the invention, the inhibitory polynucleotide is an antisense molecule.

The present invention also provides isolated DNA molecules that encode the inhibitory polynucleotides of the invention, e.g., as described herein. In one embodiment of the invention, the DNA molecule is operably linked to at least one expression control sequence. The present invention also provides a host cell transformed or transfected with such DNA molecules that encode the inhibitory polynucleotides of the invention. Further, the invention also provides a microorganism that contains a DNA molecule(s) that encodes an inhibitory polynucleotide of the invention. In one embodiment, the invention provides a nonhuman transgenic animal in which the somatic and germ cells contain DNA that encodes an inhibitory polynucleotide of the invention. In another embodiment, the invention provides a transgenic plant in which the somatic and germ cells contain DNA that that encodes an inhibitory polynucleotide of the invention.

In one embodiment of the invention, an siRNA of the invention (e.g., as described above) further comprises a second strand of that is complementary to the first strand of the siRNA. Also, the present invention provides an isolated DNA molecule that encodes a second strand of an siRNA molecule of the invention. In one embodiment of the invention, the isolated DNA molecule may be operably linked to at least one expression control sequence. The invention also provides a host cell transformed with such operably linked DNA molecule(s). In one embodiment, the invention provides a microorganism that contains DNA that encodes the second strand of an siRNA of the invention. The invention also provides a nonhuman transgenic animal in which the somatic and germ cells contain DNA that encodes a second strand of an siRNA molecule of the invention. In another embodiment, the invention provides a transgenic plant in which the somatic and germ cells contain DNA that encodes a second strand of an siRNA of the invention.

The invention also provides a kit comprising an inhibitory polynucleotide of the invention and methods of using an inhibitory polynucleotide of the invention.

The invention also provides a method of downregulating the expression of a transgene by a host cell, wherein the transgene is transcribed as part of an mRNA transcript comprising a nucleotide sequence corresponding to an IRES, the method comprising the step of introducing into the host cell an inhibitory polynucleotide that targets the IRES. In one embodiment of the invention the IRES has and/or consists essentially of the nucleotide sequence of SEQ ID NO: 1.

### BRIEF DESCRIPTION OF THE DRAWINGS

FIG. 1 shows the nucleotide sequence of the EMCV-IRES (equivalent to SEQ ID NO: 1). Indicated in bold within the EMCV-IRES sequence are examples of three portions of the EMCV-IRES sequence (IRES1, IRES2, and IRES3; SEQ ID NOs:2, 3, and 4, respectively) that may be optimally targeted by siRNA molecules. Also shown in boxes are examples of three siRNA molecules (siRNA1, siRNA2, and siRNA3; SEQ ID NOs:5, 6, and 7, respectively) that may be used to target the EMCV-IRES (at IRES1, IRES2, and IRES3, respectively).

FIG. 2A demonstrates the antibody titer (μg/ml; y-axis) produced by CHO cells genetically modified to express antibodies from a polycistronic mRNA comprising at least one EMCV-IRES sequence after transfection with the following (x-axis): control transfection reagents (control), siRNA1 molecules directed against IRES1 (IRES1), siRNA2 molecules directed against IRES2 (IRES2), siRNA3 molecules directed against IRES3 (IRES3), or a pool of siRNA1, siRNA2 and siRNA3 molecules (Pool). Bars represent the average ± SEM antibody titer of three experiments (n=3), either three days after transfection (day3; [symbol] ) or six days after transfection (day6;▪). FIG. 2B demonstrates the cell-specific productivity (titer/cell #/day; y-axis) of recombinant antibody produced by CHO cells genetically modified to express antibodies from a polycistronic mRNA comprising at least one EMCV-IRES sequence cells after transfection with the following (x-axis): control transfection reagents (control), siRNA1 molecules directed against IRES1 (IRES1), siRNA2 molecules directed against IRES2 (IRES2), siRNA3 molecules directed against IRES3 (IRES3), or a pool of siRNA1, siRNA2 and siRNA3 molecules (Pool). Bars represent the average ± SEM antibody titer of three experiments (n=3), either three days after transfection (day3; [symbol] ) or six days after transfection (day6;▪).

### DETAILED DESCRIPTION OF THE INVENTION

The present invention relates to the discovery of internal ribosome entry sites (IRESes) and the subsequent use of IRESes in recombinant DNA technology to initiate and control the translation of a protein-coding region within a polycistronic mRNA transcript. The invention is based on the discovery that targeting a targeted IRES with inhibitory polynucleotides efficiently prevents translation of at least the protein-coding region upstream of the targeted IRES, and perhaps all protein-coding regions of the mRNA transcript comprising a nucleotide sequence corresponding to the targeted IRES. Consequently, provided herein are inhibitory polynucleotides directed toward an IRES (i.e., a targeted IRES) and methods of using them in nonspecific approaches to knock down the expression of a gene of interest. As it is the IRES that is being targeted, this method does not require directing inhibitory polynucleotides precisely against the gene of interest; i.e., methods provided herein do not require that that the sequence of the gene of interest be known or determined, and allows the targeting molecules to be used to inhibit the expression of many transgenes. The method only requires that the protein-coding region transcribed from the gene of interest be within an mRNA transcript comprising a nucleotide sequence corresponding to a targeted IRES. In other words, it is likely that the entire mRNA transcript comprising the IRES will be targeted by an inhibitory polynucleotide of the invention, e.g., an siRNA molecule, for inhibition. For example, an siRNA molecule targeting the IRES on an mRNA transcript comprising (from 5′ to 3′) a first transgene, the IRES, and a second transgene, may be used to knock down expression of both the second and first transgenes. An mRNA transcript need not be polycistronic to be successfully targeted by an inhibitory polynucleotide of the invention. For example, an siRNA targeting an IRES sequence on an mRNA transcript that comprises only one transgene, which is either upstream or downstream of the IRES, may be used to knock down expression of the one transgene. In a preferred embodiment, the mRNA transcribed from the gene of interest, i.e., containing the protein-coding region of the gene of interest, is upstream of an IRES.

In particular, the present invention is based on the discovery that inhibitory polynucleotides directed against an IRES may inhibit translation of a protein-coding region that is part of an mRNA transcript comprising a nucleotide sequence corresponding to the targeted IRES. It will be apparent to one of skill in the art that use of such inhibitory polynucleotides directed toward an IRES allows methods of knocking down expression of a gene of interest, the protein-coding region of which is within an mRNA transcript comprising a nucleotide sequence corresponding to the targeted IRES, and that such methods do not require modification or targeting of the transgene itself. A skilled artisan will recognize that the inhibiting polynucleotides provided herein will not only enable the downregulation of a gene of interest, but also may be used in methods of screening siRNA libraries, e.g., as positive controls.

As such, the invention provides inhibitory polynucleotides that target an IRES. Additionally, the invention provides methods of modifying a host cell or organism to express inhibitory polynucleotides of the invention, and also provides such modified host cells or organisms. The invention also provides methods of using the inhibitory polynucleotides to alter the expression of genes of interest and as positive controls in screening assays, e.g., siRNA screening assays.

In accordance with the invention, a gene of interest may encode a therapeutic protein. A therapeutic protein, as used herein, is a protein or peptide that has a biological effect on a region in the body on which it acts or on a region of the body on which it remotely acts via intermediates. A therapeutic protein can be, for example, a secreted protein, such as, an antibody, an antigen-binding fragment of an antibody, a soluble receptor, a receptor fusion, a cytokine, a growth factor, an enzyme, or a clotting factor, as described in more detail herein. The above list of proteins is merely exemplary in nature, and is not intended to be a limiting recitation. One of ordinary skill in the art will understand that any protein may be used in accordance with the present invention and will be able to select the particular protein to be produced based as needed.

As used in the specification, the terms polypeptide, protein and peptide are synonymous and are used interchangeably. Accordingly, as used herein, the size of a protein, peptide or polypeptide generally comprises more than 2 amino acids. For example, a protein, peptide or polypeptide can comprise from about 2 to about 20 amino acids, from about 20 to about 40 amino acids, from about 40 to about 100 amino acids, from about 100 amino acids to about 200 amino acids, from about 200 amino acids to about 300 amino acids, and so on.

As used herein, an amino acid refers to any naturally occurring amino acid, any amino acid derivative or any amino acid mimic known in the art. In certain embodiments, the residues of the protein or peptide are sequential, without any non-amino acid interrupting the sequence of amino acid residues. In other embodiments, the sequence may comprise one or more non-amino acid moieties. In particular embodiments, the sequence of residues of the protein or peptide may be interrupted by one or more non-amino acid moieties.

As used herein, an antibody refers to any antibody-like molecule that has an antigen binding region, and includes antibody fragments such as Fab′, Fab, F(ab′)₂, single domain antibodies (DABs), Fv, scFv (single chain Fv), and the like. Techniques for preparing and using various antibody-based constructs and fragments are well known in the art. Means for preparing and characterizing antibodies are also well known in the art (see, e.g., Harlow and Lane, Antibodies: A Laboratory Manual, Cold Spring Harbor Laboratory, 1988; incorporated herein by reference in its entirety). For example, an antibody can include at least one, and preferably two full-length heavy chains, and at least one, and preferably two light chains. The term “antibody” as used herein includes an antibody fragment or a variant molecule such as an antigen-binding fragment (e.g., an Fab, F(ab′)2, Fv, a single chain Fv fragment, a heavy chain fragment (e.g., a camelid VHH) and a binding domain-immunoglobulin fusion (e.g., SMIP™).

The antibody can be a monoclonal or single-specificity antibody. The antibody can also be a human, humanized, chimeric, CDR-grafted, or in vitro-generated antibody. In yet other embodiments, the antibody has a heavy chain constant region chosen from, e.g., IgG1, IgG2, IgG3, or IgG4. In another embodiment, the antibody has a light chain chosen from, e.g., kappa or lambda. In one embodiment, the constant region is altered, e.g., mutated, to modify the properties of the antibody (e.g., to increase or decrease one or more of: Fc receptor binding, antibody glycosylation, the number of cysteine residues, effector cell function, or complement function). Typically, the antibody specifically binds to a predetermined antigen, e.g., an antigen associated with a disorder, e.g., a neurodegenerative, metabolic, inflammatory, autoimmune, and/or malignant disorder.

Small Modular ImmunoPharmaceuticals (SMIP™) provide an example of a variant molecule comprising a binding domain polypeptide. SMIPs and their uses and applications are disclosed in, e.g., U.S. Published Patent Application. Nos. 2003/0118592, 2003/0133939, 2004/0058445, 2005/0136049, 2005/0175614, 2005/0180970, 2005/0186216, 2005/0202012, 2005/0202023, 2005/0202028, 2005/0202534, and 2005/0238646, and related patent family members thereof, all of which are hereby incorporated by reference herein in their entireties.

Single domain antibodies can include antibodies whose complementary determining regions are part of a single domain polypeptide. Examples include, but are not limited to, heavy chain antibodies, antibodies naturally devoid of light chains, single domain antibodies derived from conventional four-chain antibodies, engineered antibodies and single domain scaffolds other than those derived from antibodies. Single domain antibodies may be any of the art, or any future single domain antibodies. Single domain antibodies may be derived from any species including, but not limited to mouse, human, camel, llama, goat, rabbit, and bovine. According to one aspect of the invention, a single domain antibody as used herein is a naturally occurring single domain antibody known as heavy chain antibody devoid of light chains. Such single domain antibodies are disclosed in International Published Application No. WO 9404678, for example. For reasons of clarity, this variable domain derived from a heavy chain antibody naturally devoid of light chain is known herein as a VHH or nanobody to distinguish it from the conventional VH of four-chain immunoglobulins. Such a VHH molecule can be derived from antibodies raised in Camelidae species, for example in camel, llama, dromedary, alpaca and guanaco. Other species besides Camelidae may produce heavy chain antibodies naturally devoid of light chain; such VHHs are within the scope of the invention.

Examples of binding fragments encompassed within the term “antigen-binding fragment” of an antibody include (i) a Fab fragment, a monovalent fragment consisting of the VL, VH, CL and CH1 domains; (ii) a F(ab′)2 fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment, which consists of a VH domain; (vi) a camelid or camelized variable domain, e.g., a VHH domain; (vii) a single chain Fv (scFv); (viii) a bispecific antibody; and (ix) one or more fragments of an immunoglobulin molecule fused to an Fc region. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see, e.g., Bird et al. (1988) Science 242:423-26; Huston et al. (1988) Proc. Natl. Acad. Sci. U.S.A. 85:5879-83). Such single chain antibodies are also intended to be encompassed within the term “antigen-binding fragment” of an antibody. These antibody fragments are obtained using conventional techniques known to those skilled in the art, and the fragments are evaluated for function in the same manner as are intact antibodies.

Other than “bispecific” or “bifunctional” antibodies, an antibody is understood to have each of its binding sites identical. A “bispecific” or “bifunctional” antibody is an artificial hybrid antibody having two different heavy/light chain pairs and two different binding sites. Bispecific antibodies can be produced by a variety of methods including fusion of hybridomas or linking of Fab′ fragments (see, e.g., Songsivilai and Lachmann (1990) Clin. Exp. Immunol. 79:315-21; Kostelny et al. (1992) J. Immunol. 148:1547-53).

Target Sequences

The invention may be applied to most, if not all, well-known IRESes (particularly those routinely used in recombinant DNA methods), without undue experimentation. Thus, it is part of the invention that a target sequence related to the invention is an IRES sequence derived from any viral or cellular gene. The sequences of most IRESes are available from public databases, e.g., www.ncbi.nlm.nih.gov, www.rangueil.inserm.fr/IRESdatabase, etc. As a nonlimiting example, the present invention relates to the use of an IRES isolated from the encephalomyocarditis virus (EMCV) genome. As such, in one embodiment, the present invention relates to isolated and purified polynucleotides of the EMCV-IRES.

The nucleotide sequence of a cDNA encoding EMCV-IRES is set forth in SEQ ID NO:1. Polynucleotides related to the present invention also include polynucleotides that hybridize under stringent conditions to SEQ ID NO:1, or complements thereof, and/or encode mRNAs that retain substantial biological activity of EMCV-IRES. Polynucleotides related to the present invention also include continuous portions of the sequence set forth in SEQ ID NO:1 comprising at least about 15 to 30 nucleotides, e.g., 19-27 nucleotides. In one embodiment, polynucleotides related to the present invention also include continuous portions of the sequence set forth in SEQ ID NO:1 comprising about 19 or 21 consecutive nucleotides.

The isolated polynucleotides related to the present invention (e.g., SEQ ID NO:1, complements thereof, and continuous portions thereof) may be used as hybridization probes and primers to identify and isolate nucleic acids having sequences identical to, or similar to, those encoding the disclosed polynucleotides. Hybridization methods for identifying and isolating nucleic acids include polymerase chain reaction (PCR), Southern hybridization, and Northern hybridization, and are well known to those skilled in the art.

Hybridization reactions may be performed under conditions of different stringencies. The stringency of a hybridization reaction includes the difficulty with which any two nucleic acid molecules will hybridize to one another. Preferably, each hybridizing polynucleotide hybridizes to its corresponding polynucleotide under reduced stringency conditions, more preferably stringent conditions, and most preferably highly stringent conditions. Examples of stringency conditions are shown in Table 1 below: highly stringent conditions are those that are at least as stringent as, for example, conditions A-F; stringent conditions are at least as stringent as, for example, conditions G-L; and reduced stringency conditions are at least as stringent as, for example, conditions M-R.

TABLE 1

| Strin- | | | | |
|---|---|---|---|---|
| gency | Poly- | Hybrid | Hybridization | Wash |
| Condi- | nucleotide | Length | Temperature and | Temperature |
| tion | Hybrid | (bp)¹ | Buffer² | and Buffer² |
| A | DNA:DNA | >50 | 65° C.; 1X SSC -or- | 65° C.; |
| | | | 42° C.; 1X SSC, | 0.3X SSC |
| | | | 50% formamide | |
| B | DNA:DNA | <50 | T_{B}*; 1× SSC | T_{B}*; 1× SSC |
| C | DNA:RNA | >50 | 67° C.; 1X SSC -or- | 67° C.; |
| | | | 45° C.; 1X SSC, | 0.3X SSC |
| | | | 50% formamide | |
| D | DNA:RNA | <50 | T_{D}*; 1× SSC | T_{D}*; 1× SSC |
| E | RNA:RNA | >50 | 70° C.; 1X SSC -or- | 70° C.; |
| | | | 50° C.; 1X SSC, | 0.3xSSC |
| | | | 50% formamide | |
| F | RNA:RNA | <50 | T_{F}*; 1× SSC | T_{f}*; 1× SSC |
| G | DNA:DNA | >50 | 65° C.; 4X SSC -or- | 65° C.; |
| | | | 42° C.; 4X SSC, | 1X SSC |
| | | | 50% formamide | |
| H | DNA:DNA | <50 | T_{H}*; 4× SSC | T_{H}*; 4× SSC |
| I | DNA:RNA | >50 | 67° C.; 4X SSC -or- | 67° C.; |
| | | | 45° C.; 4X SSC, | 1X SSC |
| | | | 50% formamide | |
| J | DNA:RNA | <50 | T_{J}*; 4× SSC | T_{J}*; 4× SSC |
| K | RNA:RNA | >50 | 70° C.; 4X SSC -or- | 67° C.; |
| | | | 50° C.; 4X SSC, | 1X SSC |
| | | | 50% formamide | |
| L | RNA:RNA | <50 | T_{L}*; 2× SSC | T_{L}*; 2× SSC |
| M | DNA:DNA | >50 | 50° C.; 4X SSC -or- | 50° C.; |
| | | | 40° C.; 6X SSC, | 2X SSC |
| | | | 50% formamide | |
| N | DNA:DNA | <50 | T_{N}*; 6× SSC | T_{N}*; 6× SSC |
| O | DNA:RNA | >50 | 55° C.; 4X SSC -or- | 55° C.; |
| | | | 42° C.; 6X SSC, | 2X SSC |
| | | | 50% formamide | |
| P | DNA:RNA | <50 | T_{P}*; 6× SSC | T_{P}*; 6× SSC |
| Q | RNA:RNA | >50 | 60° C.; 4X SSC -or- | 60° C.; |
| | | | 45° C.; 6X SSC, | 2X SSC |
| | | | 50% formamide | |
| R | RNA:RNA | <50 | T_{R}*; 4× SSC | T_{R}*; 4× SSC |
| ¹The hybrid length is that anticipated for the hybridized region(s) of the hybridizing polynucleotides. When hybridizing a polynucleotide to a target polynucleotide of unknown sequence, the hybrid length is assumed to be that of the hybridizing polynucleotide. When polynucleotides of known sequence are hybridized, the hybrid length can be determined by aligning the | | | | |
| # sequences of the polynucleotides and identifying the region or regions of optimal sequence complementarity. | | | | |
| ²SSPE (1xSSPE is 0.15M NaCl, 10 mM NaH₂PO₄, and 1.25 mM EDTA, pH 7.4) can be substituted for SSC (1xSSC is 0.15M NaCl and 15 mM sodium citrate) in the hybridization and wash buffers; washes are performed for 15 minutes after hybridization is complete. | | | | |
| T_{B}*-T_{R}*: The hybridization temperature for hybrids anticipated to be less than 50 base pairs in length should be 5-10° C. less than the melting temperature (Tₘ) of the hybrid, where Tₘ is determined according to the following equations. For hybrids less than 18 base pairs in length, Tₘ(° C.) = 2(# of A + T bases) + 4(# of G + C bases). For | | | | |
| # hybrids between 18 and 49 base pairs in length, Tₘ(° C.) = 81.5 + 16.6(log₁₀ Na⁺) + 0.41(% G + C) − (600/N), where N is the number of bases in the hybrid, and Na⁺ is the concentration of sodium ions in the hybridization buffer (Na⁺ for 1xSSC = 0.165M). | | | | |

Additional examples of stringency conditions for polynucleotide hybridization are provided in Sambrook et al. (1989) Molecular Cloning: A Laboratory Manual, Chs. 9 & 11, Cold Spring Harbor Laboratory Press, Cold Spring Harbor, NY, and Ausubel et al., eds. (1995) Current Protocols in Molecular Biology, Sects. 2.10 & 6.3-6.4, John Wiley & Sons, Inc., herein incorporated by reference.

The isolated polynucleotides related to the present invention may also be used as hybridization probes and primers to identify and isolate DNAs having sequences homologous to the disclosed polynucleotides. These homologs are polynucleotides isolated from different species than those of the disclosed polynucleotides, or within the same species, but with significant sequence similarity to the disclosed polynucleotides. Preferably, polynucleotide homologs have at least 60% sequence identity; more preferably at least 75% identity; and most preferably at least 90% identity, with the disclosed polynucleotides. Preferably, homologs of the disclosed polynucleotides are those isolated from a virus, e.g., a virus of the Picornaviridae family.

The isolated polynucleotides related to the present invention may also be used as hybridization probes and primers to identify cells and tissues that express the inhibitory polynucleotides of the present invention, as described below, and the conditions under which they are expressed.

Generally, a polynucleotide according to the present invention is provided as an isolate, in isolated and/or purified form, or free or substantially free of material with which it is naturally associated, such as free or substantially free of a nucleic acid(s) flanking the sequence in a genome (e.g., a picornavirus genome), except possibly one or more regulatory sequence(s) for expression. A polynucleotide of the invention may be wholly or partially synthetic and may include genomic DNA, cDNA or RNA. Where a polynucleotide according to the invention includes RNA, reference to the sequence shown should be construed as reference to the RNA equivalent, e.g., with U substituted for T.

Inhibitory Polynucleotides

It is an object of the invention to provide inhibitory polynucleotides directed against a targeted IRES that may be used in methods of downregulating the expression of a gene of interest (e.g., endogenous gene, transgene, etc.), the transcription of which results in its protein-coding region being within an mRNA transcript comprising a nucleotide sequence corresponding to the targeted IRES, and wherein the methods do not require modification or targeting of the gene of interest itself. It is another object of the invention to provide methods of screening inhibitory polynucleotide libraries using the inhibitory polynucleotides of the invention as positive controls. To this end, the inventors have demonstrated that inhibited (i.e., reduced, interfered with, downregulated, knocked down, etc.) expression of a transgene of interest may be achieved in a cell or organism through the use of inhibitory polynucleotides, e.g., siRNA molecules, that target (e.g., bind and/or cleave) IRES mRNA (e.g., EMCV-IRES mRNA), thus preventing translation of any protein-coding region found on the same mRNA transcript as the IRES mRNA.

Altered expression of the IRES sequences related to the invention in a cell or organism may be achieved through the use of various inhibitory polynucleotides, such as antisense polynucleotides, ribozymes that bind and/or cleave the mRNA transcribed from the genes of the invention, triplex-forming oligonucleotides that target regulatory regions of the genes, and short interfering RNA that causes sequence-specific degradation of target mRNA (e.g., Galderisi et al. (1999) J. Cell. Physiol. 181:251-57; Sioud (2001) Curr. Mol. Med. 1:575-88; Knauert and Glazer (2001) Hum. Mol. Genet. 10:2243-51; Bass (2001) Nature 411:428-29).

The inhibitory antisense or ribozyme polynucleotides of the invention can be complementary to an entire coding strand of an IRES sequence related to the invention, or to only a portion thereof. Alternatively, inhibitory polynucleotides can be complementary to a noncoding region of the coding strand of an IRES sequence related to the invention. The inhibitory polynucleotides of the invention can be constructed using chemical synthesis and/or enzymatic ligation reactions using procedures well known in the art. The nucleoside linkages of chemically synthesized polynucleotides can be modified to enhance their ability to resist nuclease-mediated degradation, as well as to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman (2002) Dev. Biol. 243:209-14; Mickelfield (2001) Curr. Med. Chem. 8:1157-79). Alternatively, antisense molecules can be produced biologically using an expression vector into which a polynucleotide of the present invention has been subcloned in an antisense (i.e., reverse) orientation.

In yet another embodiment, the antisense polynucleotide molecule of the invention is an α-anomeric polynucleotide molecule. An α-anomeric polynucleotide molecule forms specific double-stranded hybrids with complementary RNA in which, contrary to the usual β-units, the strands run parallel to each other. The antisense polynucleotide molecule can also comprise a 2′-o-methylribonucleotide or a chimeric RNA-DNA analogue, according to techniques that are known in the art.

The inhibitory triplex-forming oligonucleotides (TFOs) encompassed by the present invention bind in the major groove of duplex DNA with high specificity and affinity (Knauert and Glazer, supra). Expression of the genes of the present invention can be inhibited by targeting TFOs complementary to the regulatory regions of the genes (i.e., the promoter and/or enhancer sequences) to form triple helical structures that prevent transcription of the genes.

In one embodiment of the invention, the inhibitory polynucleotides of the present invention are short interfering RNA (siRNA) molecules (see, e.g., Galderisi et al. (1999) J. Cell Physiol. 181:251-57; Sioud (2001) Curr. Mol. Med. 1:575-88). These siRNA molecules are short (preferably 19-25 nucleotides, more preferably 19 or 21 nucleotides) double-stranded RNA molecules that cause sequence-specific degradation of the targeted mRNA. This degradation is known as RNA interference (RNAi) (e.g., Bass (2001) Nature 411:428-29). Originally identified in lower organisms, RNAi has been effectively applied to mammalian cells and has recently been shown to prevent fulminant hepatitis in mice treated with siRNA molecules targeted to Fas mRNA (Song et al. (2003) Nat. Med. 9:347-51). In addition, intrathecally delivered siRNA has recently been reported to block pain responses in two models (agonist-induced pain model and neuropathic pain model) in the rat (Dorn et al. (2004) Nucleic Acids Res. 32(5):e49).

The duplex structure of siRNA molecules of the invention may comprise one or more strands of polymerized RNA, i.e., the duplex structure may be formed by a single self-complementary RNA strand comprising a hairpin loop or two complementary strands. siRNA sequences with insertions, deletions, and single point mutations relative to the targeted sequence have also been found to be effective in inhibiting the expression of the targeted sequence (Fire et al., U.S. Pat. No. 6,506,559). Accordingly, it is preferred that siRNA molecules of the invention comprise a nucleotide sequence with substantial sequence identity to at least a portion of the mRNA corresponding to the targeted IRES. For example, the duplex region of an siRNA molecule of the invention may have greater than 90% sequence identity, and preferably 100% sequence identity, to at least of portion of the mRNA corresponding to the targeted IRES. Alternatively, substantial sequence identity may be defined as the ability of at least one strand of the duplex region of the siRNA molecule to hybridize to at least a portion of the targeted IRES under at least, e.g., stringent conditions as defined as conditions G-L in Table 1, above. In a preferred embodiment, the siRNA molecule hybridizes to at least of a portion of the targeted IRES under highly stringent conditions, e.g., those that are at least as stringent as, for example, conditions A-F in Table 1, above. Since 100% sequence identity between at least one strand of the duplex region of an siRNA molecule of the invention and at least a portion of a targeted sequence is not required, siRNAs directed toward, e.g., an IRES sequence having and/or consisting essentially of SEQ ID NO:1, may also inhibit the expression of any protein-coding region located on an mRNA transcript that comprises an IRES sequence that differs from SEQ ID NO:1 due to mutations, polymorphisms, the redundancy of the genetic code, evolutionary divergence, etc. (see, e.g., Fire et al., supra). The length of the substantially identical nucleotide sequences may be at least 10, 15, 19, 21, 23, 25, 27, 50, 100, 200, 300, 400, or 500 nucleotides, is preferably 19-27 nucleotides, and is most preferably 19 or 21 nucleotides (see Fire et al., supra).

The inhibitory polynucleotides of the invention may be designed based on criteria well known in the art (e.g., Elbashir et al. (2001) EMBO J. 20:6877-88) and/or by using well-known algorithms (e.g., publicly available algorithms). For example, the targeting portion of an inhibitory polynucleotide of the invention (e.g., the duplex region of an siRNA molecule) preferably should begin with AA (most preferred), TA, GA, or CA; an siRNA molecule of the invention preferably should comprise a sequence whereby the GC ratio is 45-55%; an siRNA molecule of the invention preferably should not contain three of the same nucleotides in a row; and an siRNA molecule of the invention preferably should not contain seven mixed G/Cs in a row. Based on these criteria, or on other known criteria (e.g., Reynolds et al. (2004) Nat. Biotechnol. 22:326-30), siRNA molecules of the present invention that target an IRES, e.g., the EMCV-IRES having and/or consisting essentially of the nucleotide sequence of SEQ ID NO:1, may be designed by one of ordinary skill in the art. For example, in one embodiment, an siRNA molecule of the invention may have and/or consist essentially of a nucleotide sequence selected from the group consisting of the nucleotide sequence of SEQ ID NO:2, the nucleotide sequence of SEQ ID NO:3, and the nucleotide sequence of SEQ ID NO:4. In this embodiment, an siRNA molecule of the invention further comprises the complement of the nucleotide sequence of SEQ ID NO:2, the complement of the nucleotide sequence of SEQ ID NO:3, and the complement of the nucleotide sequence of SEQ ID NO:4, respectively.

For example, the siRNA molecules of the present invention may be generated by annealing two complementary single-stranded RNA molecules together (Fire et al., supra) or through the use of a single hairpin RNA molecule that folds back on itself to produce the requisite double-stranded portion (Yu et al. (2002) Proc. Natl. Acad. Sci. USA 99:6047-52). The siRNA molecules may be chemically synthesized (Elbashir et al. (2001) Nature 411:494-98) or produced by in vitro transcription using single-stranded DNA templates (Yu et al., supra). Alternatively, the siRNA molecules can be produced biologically, either transiently (Yu et al., supra; Sui et al. (2002) Proc. Natl. Acad. Sci. USA 99:5515-20) or stably (Paddison et al. (2002) Proc. Natl. Acad. Sci. USA 99:1443-48), using an expression vector(s), e.g., as described below, comprising polynucleotides related to the present invention in sense and/or antisense orientation relative to their promoter. Recombinant RNA polymerase may be used for transcription in vivo or in vitro, or endogenous RNA polymerase of a modified cell may mediate transcription in vivo. Recently, reduction of levels of target mRNA in primary human cells, in an efficient and sequence-specific manner, was demonstrated using adenoviral vectors that express hairpin RNAs, which are further processed into siRNA molecules (Arts et al. (2003) Genome Res. 13:2325-32).

The inhibitory polynucleotides of the invention may be constructed using chemical synthesis and enzymatic ligation reactions including procedures well known in the art. The nucleoside linkages of chemically synthesized polynucleotides may be modified to enhance their ability to resist nuclease-mediated degradation, avoid a general panic response in some organisms that is generated by duplex RNA, and/or to increase their sequence specificity. Such linkage modifications include, but are not limited to, phosphorothioate, methylphosphonate, phosphoroamidate, boranophosphate, morpholino, and peptide nucleic acid (PNA) linkages (Galderisi et al., supra; Heasman, supra; Micklefield, supra).

As described above, the isolated polynucleotides, or continuous portions thereof, related to the present invention may be operably linked in sense or antisense orientation to an expression control sequence and/or ligated into an expression vector for recombinant expression of the inhibitory polynucleotides (e.g., siRNA molecules) of the invention. General methods of recombinant expression of inhibitory polynucleotides are well known in the art.

An expression vector, as used herein, is intended to refer to a nucleic acid molecule capable of transporting another nucleic acid to which it has been linked. One type of vector is a plasmid, which refers to a circular double stranded DNA loop into which additional DNA segments may be ligated. Another type of vector is a viral vector, wherein additional DNA segments may be ligated into the viral genome. Certain vectors are capable of autonomous replication in a host cell into which they are introduced (e.g., bacterial vectors having a bacterial origin of replication and episomal mammalian vectors). Other vectors (e.g., nonepisomal mammalian vectors) can be integrated into the genome of a host cell upon introduction into the host cell, and thereby are replicated along with the host genome. Moreover, certain vectors are capable of directing the expression of the inhibitory polynucleotides to which they are operably linked. Such vectors are referred to herein as recombinant expression vectors (or simply, expression vectors). In general, expression vectors of utility in recombinant DNA techniques are often in the form of plasmids. In the present specification, plasmid and vector may be used interchangeably as the plasmid is the most commonly used form of vector. However, the invention is intended to include other forms of expression vectors, such as viral vectors (e.g., replication defective retroviruses, adenoviruses and adeno-associated viruses) that serve equivalent functions.

A person of ordinary skill in the art will know how to create an expression vector from which an inhibitory polynucleotide of the invention may be transcribed. First, a skilled artisan will know that a regulatory region (e.g., promoter, enhancer, silencer, splice donor, acceptor, etc.) may be used to transcribe an RNA strand or RNA strands of an inhibitory polynucleotide of the invention from an expression construct. Second, a skilled artisan will recognize that, e.g., in creating a duplex siRNA molecule of the invention, the sense and antisense strands of the targeted portion of the targeted IRES may be transcribed as two separate RNA strands that will anneal together, or as a single RNA strand that will form a hairpin loop and anneal with itself. For example, a skilled artisan will know how to create an expression construct whereby the targeted portion of a targeted IRES is inserted between two promoters (e.g., two bacteriophage T7 promoters, or two different promoters) such that transcription occurs bidirectionally and will result in complementary RNA strands that may subsequently anneal to form an inhibitory siRNA of the invention. Alternatively, a targeted portion of a targeted IRES may exist as a first and second coding region together on a single expression vector, wherein the first coding region of the targeted portion of a targeted IRES is in sense orientation relative to its controlling promoter, and wherein the second coding region of the targeted portion of a targeted IRES is in antisense orientation relative to its controlling promoter. A skilled artisan will recognize that if transcription of the sense and antisense coding regions of the targeted portion of the targeted IRES occurs from two separate promoters, the result will be two separate RNA strands that may subsequently anneal to form an inhibitory siRNA of the invention. On the other hand, if transcription of the sense and antisense targeted portion of the targeted IRES is controlled by a single promoter, the resulting transcript will be a single hairpin RNA strand that is self-complementary, i.e., forms a duplex by folding back on itself to create an siRNA molecule of the invention. In the latter configuration, a skilled artisan will also recognize that a spacer, e.g., of nucleotides, between the sense and antisense coding regions of the targeted portion of the targeted IRES will improve the ability of the single strand RNA to form a hairpin loop, wherein the hairpin loop comprises the spacer. In a preferred embodiment, the spacer comprises a length of nucleotides of at least about 5, 9, 11, or 15 nucleotides. Finally, a skilled artisan will recognize that the sense and antisense coding regions of the targeted portion of the targeted IRES may each be on a separate expression vector and under the control of its own promoter.

The recombinant expression vectors of the invention may carry additional sequences, such as sequences that regulate replication of the vector in host cells (e.g., origins of replication) and selectable marker genes. The selectable marker gene facilitates selection of host cells into which the vector has been introduced. For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr⁻ host cells with methotrexate selection/amplification) and the neo gene (for G418 selection).

Suitable vectors may be chosen or constructed, containing appropriate regulatory sequences, including promoter sequences, terminator sequences, polyadenylation sequences, enhancer sequences, marker genes and other sequences, e.g., sequences that regulate replication of the vector in the host cells (e.g., origins of replication) as appropriate. Vectors may be plasmids or viral, e.g., phage, or phagemid, as appropriate. For further details see, for example, Molecular Cloning: a Laboratory Manual: 2nd ed., Sambrook et al., Cold Spring Harbor Laboratory Press, 1989. Many known techniques and protocols for manipulation of nucleic acid, for example, in preparation of nucleic acid constructs, mutagenesis, sequencing, introduction of DNA into cells and gene expression, and analysis of proteins, are described in detail in Current Protocols in Molecular Biology, 2nd ed., Ausubel et al. eds., John Wiley & Sons, 1992.

In one embodiment, a recombinant vector of the invention comprises the EMCV-IRES having and/or consisting essentially of the nucleotide sequence of SEQ ID NO:1 and its complement, or continuous portions thereof, for the transcription of the inhibitory polynucleotides of the invention as described above. For example, an expression vector of the invention may comprise one or two copies of double-stranded DNA, wherein the first DNA strand comprises a nucleotide sequence selected from the group consisting of the nucleotide sequence of nucleotides 27-46 of SEQ ID NO:1, the nucleotide sequence of nucleotides 347-366 of SEQ ID NO:1, the nucleotide sequence of nucleotides 472-491 of SEQ ID NO:1, and subsequences or portions thereof, and wherein the second DNA strand comprises a nucleotide sequence complementary to the nucleotide sequence of the first DNA strand. A skilled artisan will recognize that such a construct may produce an inhibitory polynucleotide of the invention having or consisting essentially of a nucleotide sequence selected from the group consisting of the nucleotide sequence of SEQ ID NO:2, the nucleotide sequence of SEQ ID NO:3, the nucleotide sequence of SEQ ID NO:4, and subsequences thereof, respectively. Thus, nucleotides 27-46 of SEQ ID NO:1 (i.e., SEQ ID NO:2), nucleotides 347-366 of SEQ ID NO:1 (i.e., SEQ ID NO:3), and nucleotides 472-491 of SEQ ID NO:1 (i.e., SEQ ID NO:4) represent exemplary siRNA target sites.

Host Cells/Organisms

A further aspect of the present invention provides a method of modifying a host cell or organism with an inhibitory polynucleotide of the invention. Additionally, the present invention provides a host cell or organism comprising an inhibitory polynucleotide as disclosed herein.

A number of cell lines may act as suitable host cells for introduction or recombinant expression of the inhibitory polynucleotides of the present invention. The inhibitory polynucleotides of the present invention (or expression vector(s) from which the inhibitory polynucleotide of the invention is transcribed) may be introduced into, e.g., a cell line derived from plant or animal tissue. One of skill in the art will recognize that an inhibitory polynucleotide of the invention (or expression vector(s) from which an inhibitory polynucleotide of the invention is transcribed) is preferably introduced into a host cell that comprises an IRES polynucleotide related to the invention, e.g., SEQ ID NO:1, and more preferably into a host cell that has been modified to comprise an IRES polynucleotide related to the present invention. A skilled artisan will recognize that, as part of the invention, mammalian host cells should be modified to comprise a viral IRES polynucleotide related to the invention to prevent the inadvertent inhibition of endogenous genes when an inhibitory polynucleotide of the invention targeting the IRES polynucleotide is introduced to the modified host cell. In the case where the host cell is not derived from a mammalian cell, IRES sequences derived from mammalian genes may be preferable. Although these are preferred embodiments, a skilled artisan will also recognize that an inhibitory polynucleotide of the invention (or expression vector(s) from which an inhibitory polynucleotide of the invention is transcribed) may be introduced into host cells not comprising an IRES polynucleotide related to the invention, e.g., for control purposes.

Mammalian host cell lines include, for example, COS cells, CHO cells, 293 cells, A431 cells, 3T3 cells, CV-1 cells, HeLa cells, L cells, BHK21 cells, HL-60 cells, U937 cells, HaK cells, Jurkat cells, as well as cell strains derived from in vitro culture of primary tissue and primary explants. Plant host cell lines include, but are not limited to, corn, tobacco, Arabidopsis, rapeseed, and Lemna plant cells.

Alternatively, it may be possible to recombinantly express the inhibitory polynucleotides of the present invention in lower eukaryotes such as yeast or in prokaryotes. Potentially suitable yeast strains include Saccharomyces cerevisiae, Schizosaccharomyces pombe, Kluyveromyces strains, and Candida strains. Potentially suitable bacterial strains include Escherichia coli, Bacillus subtilis, and Salmonella typhimurium.

The inhibitory polynucleotides of the present invention may also be recombinantly expressed by operably linking the isolated polynucleotides of the present invention to suitable control sequences in one or more insect expression vectors, such as baculovirus vectors, and employing an insect cell expression system. Materials and methods for baculovirus/Sf9 expression systems are commercially available in kit form (e.g., the MAXBAC® kit, Invitrogen, Carlsbad, Calif.).

Any available technique for the introduction of the inhibitory polynucleotides of the invention (or expression vector(s) from which the inhibitory polynucleotides are transcribed) into host cells or organisms will be well known by one of ordinary skill in the art and may be used.

For example, if synthesized chemically or by in vitro enzymatic synthesis, the inhibitory polynucleotides of the invention may be purified prior to introduction into a host cell or organism. For example, RNA may be purified from a mixture by extraction with a solvent or resin, precipitation, electrophoresis, chromatography, or a combination thereof. Alternatively, the RNA may be used with no or a minimum of purification to avoid losses due to sample processing. The RNA may be dried for storage or dissolved in an aqueous solution. The solution may contain buffers or salts to promote annealing, and/or stabilization of the duplex strands. After purification, the inhibitory polynucleotides of the invention (or expression vector(s) from which the inhibitory polynucleotides are transcribed), may be directly introduced into the cell, introduced extracellularly into a cavity or interstitial space or into the circulation of an organism, introduced orally, or introduced by bathing a cell or organism in a solution comprising the inhibitory polynucleotides of the invention. Physical methods of introducing nucleic acids include injection of a solution comprising the inhibitory polynucleotides of the invention, bombardment by particles covered by the inhibitory polynucleotides, soaking or bathing the cell or organism in the solution, or electorporation.

For eukaryotic cells, suitable techniques for the introduction of an expression vector(s) that encode for an inhibitory polynucleotide of the invention may include calcium phosphate transfection, DEAE-Dextran, electroporation, liposome-mediated transfection, and transduction using retrovirus or other viruses, e.g., vaccinia or, for insect cells, baculovirus. In a preferred embodiment, a viral construct packaged into a viral particle would accomplish both efficient introduction of an expression construct(s) of the invention into the cell and transcription of the inhibitory polynucleotides of the invention that is encoded by the expression construct(s). For bacterial cells, suitable techniques may include calcium chloride transformation, electroporation and transfection using bacteriophage. A skilled artisan will recognize that for plant cells, well-known techniques similar to those used for eukaryotic cells (e.g., Agrobacterium-mediated transformation and “gene gun” methods using gold particles to physically introduce plasmid DNA into plant tissue) may be used. Additionally, the inhibitory polynucleotides of the invention may be introduced along with components that perform one or more of the following activities: enhance uptake by the cell, promote annealing of duplex strands, stabilize the hybridization of annealed strands, or otherwise increase targeting of the targeted IRES. Finally, the introduction may be followed by causing or allowing expression from the nucleic acid, e.g., by culturing host cells under conditions for expression of the gene.

Expression of an inhibitory polynucleotide of the present invention in an organism may also be achieved through the creation of nonhuman transgenic plants or animals into whose genomes IRES polynucleotides related to the present invention, or continuous portions thereof, have been introduced. Such transgenic plants or animals include those that have multiple copies of an inhibitory polynucleotide of the present invention. A tissue-specific regulatory sequence(s) may be operably linked to an IRES polynucleotide, or continuous portion thereof, to direct expression of an inhibitory polynucleotide of the present invention to particular cells or a particular developmental stage. Methods for generating transgenic plants (e.g., via physical introduction of the inhibitory nucleotide) or for generating transgenic animals (e.g., via embryo manipulation and microinjection, including, but not limited to, animals such as mice, goats, nematodes, etc.) have become conventional and are well known in the art (e.g., Ma et al. (1995) Science 268:716-19; Smith and Glick (2000) Biotechnol. Adv. 18:85-89; Peeters et al. (2001) Vaccine 19:2756-61; Bockamp et al. (2002) Physiol. Genomics 11:115-32).

Methods of the Invention

Instead of the time-consuming and laborious isolation of mutants by traditional genetic screening, the function of uncharacterized genes may be determined by employing inhibitory polynucleotides of the invention to inhibit the expression of a gene of interest (e.g., endogenous gene, transgene, etc.). Such inhibition of expression may be used, e.g., to reduce the amount and/or to alter the timing of the activity of the gene of interest. The invention may be used in determining potential targets for pharmaceutics, understanding normal and pathological events associated with development, determining signaling pathways responsible for postnatal development/aging, and the like. As a nonlimiting example, a simple assay would be to modify a host cell to express a transgene of interest (of known or unknown function) such that the protein-coding region is transcribed within an mRNA transcript comprising a sequence corresponding to a targeted IRES, and then to use the inhibitory polynucleotides of the invention that target the targeted IRES to inhibit (reduce, downregulate, knock down, suppress, etc.) the expression of the transgene of interest. In another nonlimiting embodiment, the inhibitory polynucleotides of the invention are used as positive controls in screening assays, e.g., siRNA screening assays.

Inhibition of expression refers to an observable decrease in the level of gene products (e.g., mRNA and/or protein), and may be detected by examination of the outward properties of the host cell or organism, or by biochemical techniques such as hybridization reactions (e.g., Northern blot analysis, RNase protection assays, microarray analysis, etc.), reverse transcription and polymerase chain reactions, binding reactions (e.g., Western blots, ELISA, FACS, etc.), reporter assays, drug resistance assays, etc. Depending on the method of detection, greater than 5%, 10%, 33%, 50%, 90%, 95% or 99% inhibition of the expression of a gene of interest by a host cell or organism treated with an inhibitory polynucleotide of the invention compared to a nontreated host cell or organism may be expected. Additionally, treatment of a population of host cells according to a method provided herein may result in a fraction of the cells (e.g., at least 2%, 5%, 10%, 20%, 50%, 75%, 90%, 95%, or 99% of treated cells) exhibiting inhibited expression of a gene of interest. Increasing the dose of inhibitory polynucleotides may increase the amount of inhibition detected. A skilled artisan will recognize that since the inhibitory polynucleotides are directed against a targeted IRES, and not a gene of interest, quantitation of expression of the gene of interest in treated cell(s) or organism(s) may show dissimilar levels of inhibition at the mRNA level compared to the protein level. As an example, although the efficiency of inhibition may be determined by detecting the mRNA level of the gene of interest, e.g., by Northern blot analysis, a preferred method of determining the level of inhibition is by detecting the level of protein.

The inhibitory polynucleotides of the invention may be introduced into a host cell or organism, as described above, in sufficient amounts to allow introduction of at least one copy of an inhibitory polynucleotide into the cell. Higher doses (e.g., at least 5, 10, 100, 500, or 1000 copies per cell) of an inhibitory polynucleotide may yield more effective inhibition.

Downregulation of a Transgene of Interest

The present invention provides a method of inhibiting the expression of a transgene of interest, the protein-coding region of which is transcribed by a host cell or organism within an mRNA transcript comprising a nucleotide sequence corresponding to a targeted IRES. The method comprises introducing an inhibitory polynucleotide of the invention that targets a targeted IRES into the host cell or organism comprising the transgene of interest, wherein the transgene is transcribed into an mRNA transcript comprising a sequence corresponding to the targeted IRES. A skilled artisan will recognize that although the inhibitory molecules of the invention specifically target the IRES, introduction of the inhibitory polynucleotides of the invention will also result in downregulation of any protein-coding region located on the same mRNA transcript as the IRES.

Thus, the inhibitory polynucleotides of the invention are particularly useful because they may be used to inhibit the expression of more than the targeted IRES, i.e., they may be used to knock down expressions of transgenes with nucleotide sequences that differ, i.e., do not correspond to the sequence of the inhibitory polynucleotides (see Example 1). Any transgene may be inhibited using the inhibitory polynucleotides of the invention as long as transcription of the transgene results in its protein-coding region being within an mRNA transcript comprising a nucleotide sequence corresponding to an IRES sequence related to the invention.

Screening Assays

As described above, the inhibitory polynucleotides of the present invention that target a targeted IRES may be used to inhibit the expression of a gene, e.g., a transgene that is transcribed as part of an mRNA transcript comprising a sequence corresponding to the targeted IRES. In at least one other embodiment, the inhibitory polynucleotides of the invention are used as positive controls in methods of screening libraries of inhibitory polynucleotides directed toward a particular gene (including endogenous genes, transgenes, etc.).

The inhibitory polynucleotides of the invention are useful as positive controls in methods of screening a library of inhibitory polynucleotides, e.g., siRNA molecules, for inhibitory polynucleotides that optimally inhibit the expression of a gene of interest. For high throughput assays, a positive control on each assay plate may be used to validate the results from each plate.

For example, a transgene of interest could be placed into an expression vector such that its protein-coding region is transcribed as part of an mRNA transcript comprising a nucleotide sequence corresponding to a targeted IRES. The expression vector may then be used to modify a host cell. Modified host cells may then be subjected to a library of inhibitory polynucleotides directed against the transgene of interest, wherein the library comprises as a positive control at least one inhibitory polynucleotide of the invention that targets the targeted IRES.

In another embodiment of the invention, the inhibitory polynucleotides of the invention are used as positive controls to screen, or optimize the screening of, a library of the inhibitory polynucleotides directed against an endogenous gene of interest. For example, a host cell may be modified with an expression vector comprising a reporter nucleic acid, wherein the protein-coding region of the reporter nucleic acid is part of an mRNA transcript comprising a nucleotide sequence corresponding to a targeted IRES. In this embodiment, an inhibitory polynucleotide(s) of the invention is useful as a positive control(s) by inhibiting the expression of the reporter nucleic acid. Detection of such inhibition of reporter nucleic acid activity via well-known reporter assays serves as validation of the screening protocol.

Use of the inhibitory polynucleotides of the invention in methods of downregulating the expression of a reporter nucleic acid may be useful for screening a library of inhibitory polynucleotides directed against an endogenous gene of interest, and/or for screening a library of transgenes for sequences that may induce novel phenotypes. The function of the inhibitory polynucleotides in the latter method originates from the ability of the inhibitory polynucleotides of the invention to downregulate the expression of any gene that is transcribed into an mRNA transcript comprising a sequence that corresponds to an IRES. For example, a library may comprise a plurality of expression vectors, wherein each expression vector comprises a unique transgene sequence, an IRES, and a reporter nucleic acid, such that each will be transcribed into the same one polycistronic mRNA. The library may then be used to modify a plurality of a host cell, wherein each host cell of the plurality is modified with a different expression vector. The phenotype of each modified host cell may then be observed. The transgene producing a phenotype of interest may then be further analyzed (e.g., its expression may be inhibited) using the inhibitory polynucleotides of the invention, wherein downregulation of the reporter nucleic acid will serve as a positive indication that an observed reverse in phenotype is correlated with downregulation of the transgene.

In the above-described assays, many of the well-known reporter nucleic acids and related assays may be used. In one embodiment, the reporter nucleic acid is green fluorescent protein. In a second embodiment, the reporter is β-galactosidase. In other embodiments, the reporter nucleic acid is alkaline phosphatase, β-lactamase, luciferase, or chloramphenicol acetyltransferase.

The present invention may be used alone, or as a component of a kit having at least one of the reagents necessary to carry out the introduction of the inhibitory polynucleotides of the invention to test samples, i.e., host cells or organisms. Such a kit may also include instructions to allow a user of the kit to practice the invention.

The entire contents of all references, patents, and patent applications cited throughout this application are hereby incorporated by reference herein.

### EXAMPLE

The following Example provides illustrative embodiments of the invention and does not in any way limit the invention. One of ordinary skill in the art will recognize that numerous other embodiments are encompassed within the scope of the invention.

### Example 1

### Knocking Down Gene Expression Using siRNA Directed Against the EMCV-IRES

### Example 1.1

### Materials and Methods

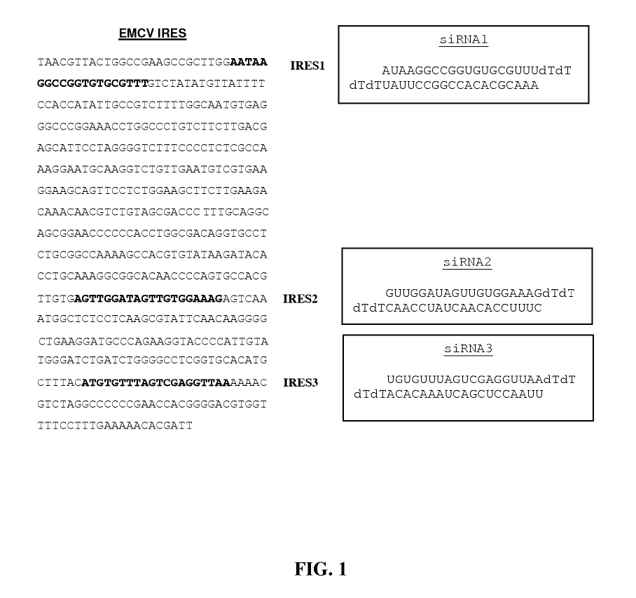

The publicly available Dharmacon siRNA design algorithm (see www.dharmacon.com/sidesign/; see also Reynolds et al., supra) was used to design siRNA molecules (hereinafter “siRNAs”) directed against the EMCV-IRES. Three portions of the IRES sequence were identified by the Dharmacon algorithm as the optimally targeted sequences (IRES1, IRES2, IRES3), and were chosen to be targeted by siRNA molecules (FIG. 1). In particular, siRNA1, siRNA2, and siRNA3 were synthesized by Dharmacon (Lafayette, Colo.) to target IRES1, IRES2, and IRES3, respectively (FIG. 1).

The three synthesized siRNA molecules were used to transfect a CHO cell line that was stably modified to express a recombinant antibody. The CHO cell line was modified with an expression vector that encoded the heavy chain of the antibody and an expression vector that encoded the light chain of the antibody. The expression vectors transcribed either the heavy or light chain into a polycistronic mRNA, which comprised the heavy or light chain protein-coding region upstream of a sequence that corresponds to the EMCV-IRES, and a different selectable marker downstream of the sequence that corresponds to the EMCV-IRES. It was expected that siRNA-mediated knockdown of EMCV-IRES-containing transcripts would result in knockdown of expression of the recombinant antibody (i.e., either or both the heavy and light chain genes upstream of the IRES). Such knockdown of expression is easily assessed by monitoring the expression of the recombinant monoclonal antibody in the conditioned media, e.g., via methods of western blotting, ELISA, or automated bead-based capture/detection methods (e.g., IGEN-based assays (Roche Diagnostics, Alameda, Calif.)). The modified CHO cells were transfected with each siRNA individually, or with a pool of all three siRNAs, in 72-hour and 144-hour secretion assays (n=3 each). Antibody titer and cell numbers were assessed using an IGEN-based assay at 72 hours (3 days) and 144 hours (6 days) to estimate cell-specific productivity (titer/cell number/day), which normalizes titer data for differences in seed density and cell growth during the experiment.

### Example 1.2

### Results

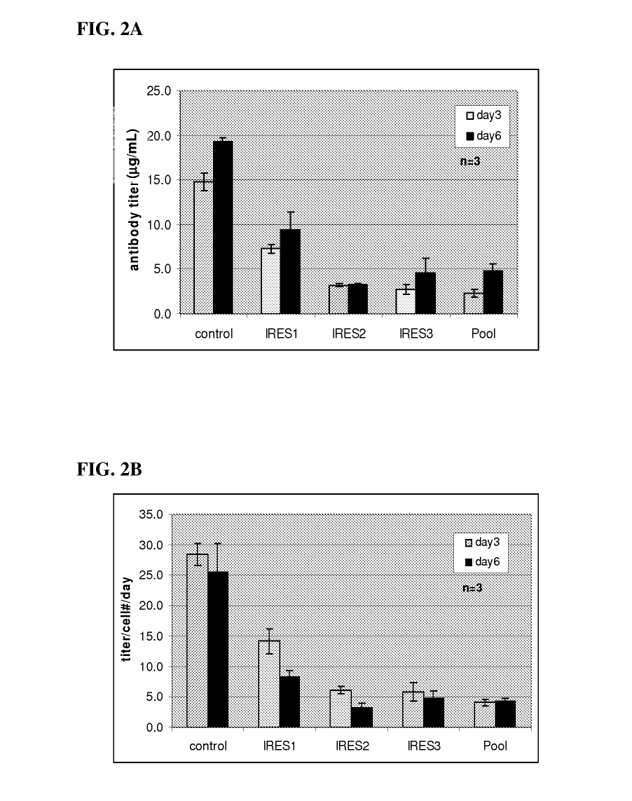

FIGS. 2A and 2B demonstrate that all three siRNAs can mediate knockdown of monoclonal antibody transgene expression in the conditioned medium. The siRNA molecules directed against IRES2 and IRES3 appear to be more effective than an siRNA molecule directed against IRES1, and the pool of siRNA molecules was very effective in knocking down expression. The knockdown was observed at both 72 hours and 144 hours post-transfection. This effect was observed in several CHO cell lines expressing different monoclonal antibodies (data not shown). The present invention demonstrates that IRES siRNAs may be used with a cell line that utilizes an IRES in the expression vector. The ability to monitor knockdown of product gene expression using a titer-based assay also eliminates the need to develop other validation assays, such as real time PCR.

What is claimed is:

1. An inhibitory polynucleotide directed against an IRES.

2. The inhibitory polynucleotide of claim 1, wherein the IRES has the nucleotide sequence of SEQ ID NO:1.

3. The inhibitory polynucleotide of claim 2, wherein the inhibitory polynucleotide comprises a first strand of an siRNA.

4. The inhibitory polynucleotide of claim 3, wherein the first strand of the siRNA has the RNA equivalent of a nucleotide sequence selected from the group consisting of the nucleotide sequence of SEQ ID NO:1, a portion of the nucleotide sequence of SEQ ID NO:1, the complement of the nucleotide sequence of SEQ ID NO:1, and a portion of the complement of the nucleotide sequence of SEQ ID NO:1.

5. The inhibitory polynucleotide of claim 4, wherein the first strand of the siRNA is between 5 and 548 nucleotides in length.

6. The inhibitory polynucleotide of claim 5, wherein the first strand of the siRNA has the RNA equivalent of a nucleotide sequence selected from the group consisting of the nucleotide sequence of SEQ ID NO:2, the nucleotide sequence of SEQ ID NO:3, the nucleotide sequence of SEQ ID NO:4, and the nucleotide sequence of subsequences thereof.

7. The inhibitory polynucleotide of claim 6, wherein the first strand of the siRNA is self complementary and further comprises a hairpin loop.

8. The inhibitory polynucleotide of claim 7, wherein the first strand of the siRNA further comprises the RNA equivalent of a nucleotide sequence selected from the group consisting of the nucleotide sequence complementary to the nucleotide sequence of SEQ ID NO:2, the nucleotide sequence complementary to the nucleotide sequence of SEQ ID NO:3, and the nucleotide sequence complementary to the nucleotide sequence of SEQ ID NO:4.

9. The inhibitory polynucleotide of claim 2, wherein the inhibitory polynucleotide is an antisense molecule.

10. The inhibitory polynucleotide of claim 6, wherein the inhibitory polynucleotide further comprises a second strand of the siRNA that is complementary to the first strand of the siRNA.

11. An isolated DNA molecule that encodes the inhibitory polynucleotide of claim 1.

12. The isolated DNA molecule of claim 11, wherein the DNA molecule is operably linked to at least one expression control sequence.

13. A host cell transformed or transfected with the isolated DNA molecule of claim 12.

14. A microorganism that contains DNA that encodes the inhibitory polynucleotide of claim 1.

15. A nonhuman transgenic animal in which the somatic and germ cells contain DNA that encodes the inhibitory polynucleotide of claim 1.

16. A transgenic plant in which the somatic and germ cells contain DNA that encodes the inhibitory polynucleotide of claim 1.

17. An isolated DNA molecule that encodes the second strand of the siRNA of claim 10.

18. The isolated DNA molecule of claim 17, wherein the DNA molecule is operably linked to at least one expression control sequence.

19. A host cell transformed or transfected with the isolated DNA molecule of claim 18.

20. A microorganism that contains DNA that encodes the second strand of the siRNA of claim 10.

21. A nonhuman transgenic animal in which the somatic and germ cells contain DNA that encodes the second strand of the siRNA of claim 10.

22. A transgenic plant in which the somatic and germ cells contain DNA that encodes the second strand of the siRNA of claim 10.

23. A kit comprising the inhibitory polynucleotide of claim 1.

24. A method of downregulating the expression of a transgene by a host cell, wherein the transgene is transcribed as part of an mRNA transcript comprising a nucleotide sequence corresponding to an IRES, the method comprising the step of introducing into the host cell an inhibitory polynucleotide that targets the IRES.

25. The method of claim 24, wherein the IRES consists essentially of the nucleotide sequence of SEQ ID NO:1.


SEQUENCE LISTING (7 sequences)

SEQ ID NO: 1 - DNA, 548 bp, Encephalomyocarditis virus
taacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaacgtctaggccccccgaaccacggggacgtggttttcctttgaaaaacacgatt

SEQ ID NO: 2 - DNA, 20 bp, Encephalomyocarditis virus
aataaggccggtgtgcgttt

SEQ ID NO: 3 - DNA, 20 bp, Encephalomyocarditis virus
agttggatagttgtggaaag

SEQ ID NO: 4 - DNA, 20 bp, Encephalomyocarditis virus
atgtgtttagtcgaggttaa

SEQ ID NO: 5 - DNA, 21 bp, Artificial
  First strand of siRNA1 (as in FIG. 1)
  misc_RNA (1)..(21)
auaaggccggugugcguuutt

SEQ ID NO: 6 - DNA, 21 bp, Artificial
  First strand of siRNA2 (as in FIG. 1)
  misc_RNA (1)..(21)
guuggauaguuguggaaagtt

SEQ ID NO: 7 - DNA, 21 bp, Artificial
  First strand of siRNA3 (as in FIG. 1)
  misc_RNA (1)..(21)
uguguuuagucgagguuaatt